(19) United States
(12) Patent Application Publication
(10) Pub. No.: US 2014/0287428 A1
(43) Pub. Date: Sep. 25, 2014

(54) METHOD FOR DETECTION OF BACTERIA IN MILK

(71) Applicant: Sietzema Sietze, Vledderveen (NL)

(72) Inventor: Sietzema Sietze, Vledderveen (NL)

(21) Appl. No.: 14/297,842

(22) Filed: Jun. 6, 2014

Related U.S. Application Data
(63) Continuation-in-part of application No. PCT/EP2012/074742, filed on Dec. 7, 2012.

(30) Foreign Application Priority Data
Dec. 9, 2011 (EP) 11192838.8

(51) Int. Cl. C12Q 1/06

(52) U.S. Cl. CPC C12Q 1/06; C12Q 2304/00
USPC 435/7.1

(57) ABSTRACT

A method of detecting the presence of bacteria in milk is provided, wherein milk is contacted with a preparation which may comprise an effective amount of at least one type of antibodies, said antibodies being capable of specifically binding to bacteria to be detected; staining said antibodies, before or after said contacting, with a staining preparation, said staining preparation being selected so that the antibodies stained therewith exhibit a color change or a change in color concentration when in contact with said bacteria; and determining the concentration of stained antibodies bound to bacteria in said milk from the presence and/or the relative intensity of the color change caused by contacting said milk with said stained antibodies.

### RELATED APPLICATIONS AND INCORPORATION BY REFERENCE

This application is a continuation-in-part application of international patent application Ser. No. PCT/EP2012/074742 filed 7 Dec. 2012, which published as PCT Publication No. WO 2013/083754 on 13 Jun. 2013, which claims benefit of European patent application Serial No. 11192838.8 filed 9 Dec. 2011.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to a method of detecting the presence of bacteria in milk.

### BACKGROUND OF THE INVENTION

Certain bacteria are causing illness, especially mastitis, in milk cows which illness may negatively affect the quality of the milk. For example, mastitis is an infectious decease in the udder or nipples of an individual cow, and mastitis bacteria will generally be present in the milk produced from such cows. Various different types of bacteria may be involved in causing mastitis, i.e. there is not just one type or strain of mastitis bacterium, but many. The types of bacteria involved in mastitis generally vary from case to case.

Since milk products are generally, especially in industrial size dairy operations, produced by mixing the milk from a multiplicity of cows, said products will contain a certain quantity of undesirable germs, e.g. mastitis-causing bacteria, when some cows are infected, even if the majority of the cows generating the milk are healthy. However, there is no a priori predictability of the types and concentrations of the bacteria cells, comprised by infected milk which ends up being mixed into the finally collected milk.

Mastitis is controlled by the government in milk samples for payment control. Too high a concentration of bacteria in the delivered milk is an indication of mastitis, such that the milk producer obtains only a lower price per quantity of delivered milk. After several deliveries with high bacterial cell count, milk delivery may actually even be forbidden.

When mastitis occurs, the veterinarians (or the milk producers) are often using a cocktail of antibiotics in order to kill and/or dismantle the bacteria, in the hope that the cocktail is covering the actually present bacterial spectrum, but without initially determining which types of bacteria are indeed involved. In the worst case, wrong antibiotics, or antibiotics not specifically suited for that particular type or types of bacteria are used one after another, or simultaneously,

This uncontrolled use of antibiotics is causing more and more bacteria mutations which show increased immunity against antibiotics. This is a dangerous situation also for public health.

### SUMMARY OF THE INVENTION

It is an important objective of the invention to create an improved method for detecting bacteria or different types of bacteria in milk, especially in a (fully or at least partly) automatic detection system. In preferred embodiments, the invention permits the selective and specific detection of bacteria or types of bacteria, and prominently among these, mastitis-causing bacteria. The invention enables such specific detection also where more than one type of bacterium is present in the milk, since it enables the detection of several different types of bacteria side by side, or simultaneously. The inventive method further makes it possible to determine the type or types of bacteria that is/are causing the infection, e. g. mastitis, in order to be able to select and dose the right antibiotics to the cow.

These and other objectives are attained by the subject matter defined in the attached claims.

More specifically, an improved method for detecting bacteria in milk incorporates the following steps:
The milk is contacted with a preparation which may comprise an effective amount of at least one type of antibodies, said antibodies being capable of specifically binding to one or more types of bacteria to be detected;
said antibodies are stained, before or after said contacting, with a staining preparation, said staining preparation being selected so that the antibodies stained therewith exhibit a difference in color concentration and/or change in color when in contact with said bacteria; and
the concentration of stained antibodies bound to bacteria in said milk is determined from the presence and, in case, the relative intensity of the color caused by contacting said milk with said stained antibodies.

These steps may be used in any suitable sequence, wherein generally, however, the step utilizing the color concentration produced by combining the stained antibodies with the bacteria will necessarily follow the step of contacting said antibodies and said bacteria.

In preferred embodiments, the inventive method involves:
Said method, wherein said antibodies are capable of specifically binding to bacteria involved in mastitis;
Said method, wherein said preparation may comprise more than one type of antibodies, and preferably may comprise more than two, more preferably more than three, even more preferably more than four and most preferably five or more different types of antibodies, each type specifically binding to a different type of bacterium or a plurality of such types.

In certain aspects of the invention, the color change and/or color concentration change is detected, qualitatively and/or quantitatively, without separating the bacteria from the milk, preferably by using a fluorescent antibody stain and a detector such as a flow cytometer.

Generally, the color change and/or color concentration change is preferably detected, qualitatively and/or quantitatively, after separating the bacteria from the milk, preferably by (micro-) filtration or centrifugation.

The color change and/or concentration change can be used to determine the type of cells, especially bacteria, in the milk, and preferably also to determine the relative amount of said cells.

In preferred embodiments, the milk is rendered substantially clear, especially by partly or fully removing (and/or dissolving) its protein and/or fatty components, prior to the color detection step.

It is generally known to detect bacteria by using specific antibodies in body tissue and fluids in the medical art. Specific conditions apply however to such known uses, which set such known methods apart from the inventive method.

### DETAILED DESCRIPTION

Mastitis results in an increased amount of bacteria per weight or volume unit of milk. The bacteria may be counted by the flow cytometer principle by use of fluorescence technique.

Fluorescence technique means in this case that the bacteria are combined (and thus, reacted) with a suitably stained antibody and the combination is passed through a flow cell where under the influence of fluorescence light, the individual cells combined with the antibodies give a light pulse. The number of light pulses is detected and is in correlation with the number of bacterial cells.

There are several groups of bacteria causing mastitis. The invention is based on the fact that specific antibodies only bind to specific types of bacteria. With this data it is possible to identify different bacteria by the use of different antibodies.

It is possible to use a single type of antibody, to detect and measure the concentration of basically a single type of bacterium. However, since there will generally be more than one type of bacterium involved, and there often is no a priori information on which kinds of bacteria will be encountered, it is preferred to add up to 5 (or even more) different sorts of antibodies into a milk sample. Preferably, these will be selected to encompass the most common types of bacteria encountered in practice.

Each group of antibodies previously are stained with a corresponding number (up to 5 or more) specific different staining solutions. E. g., using 5 different stainings makes it possible that 5 different colors are present and these can be individually detected by specific Method 1 A.

### Method 1 A

When stained antibodies are surrounding (attached to, or binding to) bacteria in a milk sample, the color of the milk (or cleared milk) is changing under the influence of the bacteria. This change of color can be detected by a specific color detector.

When the bacteria are passing through a flow cell provided with one or more different color detectors (fluorescence light), the number of bacteria can be counted based on the color change caused by the stained antibodies.

This color change is depending on the type of stained antibodies. The used antibodies may be particularly selected for that particular type of bacterium, if that is already known. It is generally known which type or types of bacteria are causing mastitis, and the choice of stained antibodies will generally be based on this knowledge. By this way it can be detected and measured which type of bacterium is causing a disease in the cow, e.g. mastitis.

### Method 1 B

Stained antibodies that bind to specific unstained bacteria in a milk sample will under influence of fluorescence light in a flow cytometer cause a light pulse. Because of the fact that several antibodies will bind to a single bacterium, there is a difference in light output pulses between a single antibody free in the milk sample and multiplicities of antibodies around the bacteria passing through the flow cytometer and thus, the bacteria can be detected by the different light pulse intensity produced.

### Method 1 C

Using the principle of antibodies bound to bacteria, as shown in Method 1 A and Method 1 B, the detection can also carried out by filtering the milk sample.

To detect the bacteria in the milk sample surrounded (attached or bound) to antibodies, the milk sample can be micro-filtered in such a way that all bacteria, surrounded or not surrounded by antibodies, will stay on the filter surface. On the filter surface, a microscope or any other detection system, fluorescence or non-fluorescence, can be used to identify or recognize the antibodies-surrounded bacteria.

The flow cytometer preferably has 5 or more different color detectors. These detectors are reacting to 5 different colours. When a flow of stained bacteria passes the microscope objective, there will be a number of pulses corresponding with the number of bacteria passing.

When between the stained bacteria, also pulses (approx. same size) with different colors are detected, this is an indication of antibody reaction on a mastitis caused by different bacteria and with that the choice of antibiotics can be made.

Although the present invention and its advantages have been described in detail, it should be understood that various changes, substitutions and alterations can be made herein without departing from the spirit and scope of the invention as defined in the appended claims.

The invention is further described by the following numbered paragraphs:
1. A method of detecting the presence of bacteria in milk, wherein milk is contacted with a preparation comprising an effective amount of at least one type of antibodies, said antibodies being capable of specifically binding to bacteria to be detected;
staining said antibodies, before or after said contacting, with a staining preparation, said staining preparation being selected so that the antibodies stained therewith exhibit a color change or a change in color concentration when in contact with said bacteria; and
determining the concentration of stained antibodies bound to bacteria in said milk from the presence and/or the relative intensity of the color change caused by contacting said milk with said stained antibodies.
2. The method of paragraph 1, wherein said antibodies are capable of specifically binding to bacteria involved in mastitis.
3. The method of paragraph 2, wherein said preparation comprises more than one type of antibodies, and preferably comprises more than two, more preferably more than three, even more preferably more than four and most preferably five or more different types of antibodies, each type specifically binding to a different type of bacteria or a plurality of such types.
4. The method of any one of the preceding paragraphs, wherein the color change is detected, qualitatively and/or quantitatively, without separating the bacteria from the milk, preferably by using a fluorescent antibody stain and a detector such as a flow cytometer.
5. The method of any one of the preceding paragraphs, wherein the color change is detected, qualitatively and/or quantitatively, after separating the bacteria from the milk, preferably by (micro)filtration or centrifugation.
6. The method of any one of the preceding paragraphs, wherein the color change is used to determine the type or types of bacteria in the milk, and preferably also to determine the relative amount (concentration) of said bacteria.
7. The method of any one of the preceding paragraphs, wherein prior to the color change detection step, the milk is rendered substantially clear, especially by partly or fully removing its protein and/or fatty components.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method of detecting the presence of bacteria in milk, wherein milk is contacted with a preparation comprising an effective amount of at least one type of antibodies, said antibodies being capable of specifically binding to bacteria to be detected;
staining said antibodies, before or after said contacting, with a staining preparation, said staining preparation being selected so that the antibodies stained therewith exhibit a color change or a change in color concentration when in contact with said bacteria; and
determining the concentration of stained antibodies bound to bacteria in said milk from the presence and/or the relative intensity of the color change caused by contacting said milk with said stained antibodies.

2. The method of claim 1, wherein said antibodies are capable of specifically binding to bacteria involved in mastitis.

3. The method of claim 2, wherein said preparation comprises more than one different type of antibodies, each type specifically binding to a different type of bacteria or a plurality of such types.

4. The method of claim 3, wherein said preparation comprises more than two different types of antibodies, each type specifically binding to a different type of bacteria or a plurality of such types.

5. The method of claim 4, wherein said preparation comprises more than three different types of antibodies, each type specifically binding to a different type of bacteria or a plurality of such types.

6. The method of claim 5, wherein said preparation comprises more than four different types of antibodies, each type specifically binding to a different type of bacteria or a plurality of such types.

7. The method of claim 6, wherein said preparation comprises more than five different types of antibodies, each type specifically binding to a different type of bacteria or a plurality of such types.

8. The method of claim 1, wherein the color change is detected, qualitatively and/or quantitatively, without separating the bacteria from the milk, preferably by using a fluorescent antibody stain and a detector such as a flow cytometer.

9. The method of claim 1, wherein the color change is detected, qualitatively and/or quantitatively, after separating the bacteria from the milk.

10. The method of claim 9, wherein the separating the bacteria from the milk is by micro-)filtration or centrifugation.

11. The method of claim 1, wherein the color change determines the type or types of bacteria in the milk.

12. The method of claim 11, wherein the color change also determines the relative amount (concentration) of said bacteria.

13. The method of claim 1, wherein prior to the color change detection step, the milk is rendered substantially clear.

14. The method of claim 13, wherein the milk is rendered substantially clear by partly or fully removing its protein and/or fatty components.
